(19) United States
(12) Patent Application Publication
(10) Pub. No.: US 2026/0042690 A1
(43) Pub. Date: Feb. 12, 2026

(54) METHOD FOR RAPID FORMATION OF DENITRIFYING GRANULAR SLUDGE AND ENHANCED DENITRIFICATION OF HIGH-NITRATE NITROGEN WASTEWATER

(71) Applicant: Tongji University, Shanghai (CN)

(72) Inventors: Yinguang CHEN, Shanghai (CN); Xuemeng ZHANG, Shanghai (CN); Xuekang CAO, Shanghai (CN); Chao LIU, Shanghai (CN); Haining HUANG, Shanghai (CN); Xinyun FAN, Shanghai (CN); Meirou WU, Shanghai (CN); Chuang CHEN, Shanghai (CN)

(73) Assignee: Tongji University, Shanghai (CN)

(21) Appl. No.: 19/346,423

(22) Filed: Sep. 30, 2025

(30) Foreign Application Priority Data
Aug. 7, 2024 (CN) 2024110735949

(51) Int. Cl. C02F 3/34; C02F 3/00; C02F 101/16; C12N 1/20

(52) U.S. Cl. CPC C02F 3/341; C02F 3/006; C12N 1/20; C02F 2101/163; C02F 2209/14; C02F 2209/16; C02F 2305/06

(57) ABSTRACT

The present invention discloses a method for the rapid formation of denitrifying granular sludge and enhanced denitrification of high-nitrate nitrogen wastewater, belonging to the technical field of wastewater treatment. The method involves colonizing the succinic acid-producing microorganism A. succinogenes into sewage sludge. By leveraging its high adhesiveness and its promoting effect on denitrifying microorganisms, the method rapidly forms denitrifying granular sludge while simultaneously enhancing the denitrification of high-nitrate nitrogen wastewater. The method is simple to operate, requires no additional pH control, and generates no secondary pollution. It enables the rapid formation of stable, full-process denitrifying granular sludge, which reduces the system's acclimation period to high-nitrate wastewater, shortens the sludge cultivation cycle, and minimizes the accumulation and discharge of intermediate products, such as nitrite, during denitrification.

### TECHNICAL FIELD

The present invention relates to the technical field of wastewater denitrification treatment, and more particularly, to a method for the rapid formation of denitrifying granular sludge and for enhancing the denitrification of high-nitrate nitrogen wastewater.

### BACKGROUND

In recent years, environmental problems such as eutrophication of water bodies caused by elevated nitrate concentrations in natural waters have become increasingly severe. Consequently, the discharge standards for wastewater treatment facilities have imposed progressively stringent requirements for the removal of nitrate nitrogen. In addition to municipal sewage, the treatment of industrial wastewater containing high concentrations of nitrate nitrogen presents a significant challenge. Wastewater discharged from industries such as explosives manufacturing, meat processing, feed production, steel smelting, fertilizer manufacturing, electronic components, and nuclear fuel production often contains high concentrations of nitrate nitrogen. For example, wastewater from a certain fertilizer plant contains 950 mg/L of nitrate nitrogen, and stainless steel pickling wastewater from a certain steel plant contains 1400 mg/L of nitrate nitrogen. Simple concentration methods for treating high-nitrate nitrogen wastewater result in even higher nitrate concentrations, failing to achieve actual removal. While chemical catalytic methods can remove nitrate nitrogen, they are difficult to adapt to complex wastewater environments, are costly, and have poor sustainability. Against the backdrop of green and low-carbon development, there is an urgent need for efficient biological denitrification technology to facilitate the treatment of high-nitrate wastewater and achieve stable and efficient removal of nitrate nitrogen under low C/N conditions. However, high-nitrate wastewater often exceeds the limits of microbial communities, leading to issues such as the collapse of the denitrification system, unstable effluent quality, and high total nitrogen in the effluent.

Granular sludge is a biological aggregate formed by the cohesion of various functional bacteria, characterized by high biomass, high biological activity, and excellent settling properties, demonstrating outstanding performance in enhancing wastewater treatment capacity and resistance to shock loads. Currently, the application of granular sludge in the field of denitrification is primarily focused on anaerobic ammonium oxidation (ANAMMOX) and aerobic granular sludge technologies. However, the formation conditions for ANAMMOX granular sludge are stringent, and the granules often disintegrate in the later stages of operation due to the scouring effect of nitrogen gas produced by the denitrification reaction. Furthermore, the high concentration of nitrite (>100 mg/L) generated during the short-cut denitrification of high-nitrate wastewater is toxic to ANAMMOX microorganisms, making it difficult for this technology to adapt to high-concentration nitrate nitrogen wastewater. Aerobic granular sludge is relatively loose and susceptible to swelling and disintegration due to external factors (such as microbial community structure, metabolism, extracellular polymeric substances, oxygen mass transfer, pH, and temperature), making it difficult to operate stably in a low C/N, high-nitrate nitrogen wastewater environment. Moreover, the formation of aerobic granular sludge often relies on filamentous or polysaccharide-accumulating bacteria, which directly compete with denitrifying microorganisms for carbon sources during the anoxic phase, thereby reducing the denitrification efficiency and making it difficult for the effluent to meet standards. This presents a challenge in the treatment of high-concentration nitrate wastewater: how to rapidly form granular sludge in a short period while maintaining both high denitrification activity and a stable granular structure to achieve stable, compliant discharge of treated high-nitrate nitrogen wastewater.

### SUMMARY

To solve the aforementioned technical problems, the present invention provides a method for the rapid formation of denitrifying granular sludge and for enhancing the denitrification of high-nitrate nitrogen wastewater. The present invention utilizes the high adhesiveness of the succinic acid-producing microorganism Actinobacillus succinogenes and its promoting effect on denitrifying microorganisms to rapidly form denitrifying granular sludge, while simultaneously enhancing the denitrification treatment effect on high-nitrate nitrogen wastewater. The method of the present invention is simple to operate, requires no additional pH control, poses no biosafety risks or secondary pollution, can rapidly form stable, full-process denitrifying granular sludge, reduces the acclimation period of the denitrification system to high-nitrate nitrogen wastewater, shortens the sludge cultivation period, and reduces the accumulation and discharge of intermediate products such as nitrite during denitrification.

A first objective of the present invention is to provide a method for the rapid formation of denitrifying granular sludge and for enhancing the denitrification of high-nitrate nitrogen wastewater, comprising the following steps:
S1. Introducing inoculated sludge and denitrifying microorganisms into a sludge acclimation and high-nitrate nitrogen wastewater denitrification treatment system;
S2. Cultivating the succinic acid-producing microorganism A. succinogenes in a sterilized medium under aerobic conditions to obtain an A. succinogenes bacterial suspension;
S3. Adding the A. succinogenes bacterial suspension prepared in step S2 to the sludge acclimation and high-nitrate nitrogen wastewater denitrification treatment system described in step S1, and carrying out phased colonization and cultivation;
S4. Gradually increasing the influent nitrate nitrogen concentration of the sludge acclimation and high-nitrate nitrogen wastewater denitrification treatment system described in step S3 in stages, wherein the particle size of the denitrifying granular sludge progressively increases. After reaching the maximum design denitrification load, maintaining the system described in step S3 in long-term stable operation at the maximum load;
S5. Maturation and stabilization of the denitrifying granular sludge, enhancing the stable and compliant discharge of the effluent from the high-nitrate nitrogen wastewater treatment.

In some embodiments of the present invention, in step S1, the high-nitrate nitrogen wastewater has a nitrate nitrogen concentration of 200-1400 mg/L, an ammonia nitrogen concentration of 5-50 mg/L, a total phosphorus concentration of 5-30 mg/L, a calcium ion concentration of 0.5-150 mg/L, a magnesium ion concentration of 0.1-7.5 mg/L, a fluoride ion concentration of 0-7.5 mg/L, an iron ion concentration of 0.01-1.5 mg/L, and a pH of 7.0-8.0;

The supplementary carbon source for denitrification is glucose; the C/N ratio is 3.8-5.5, preferably 4.5. Herein, the C/N ratio refers to the ratio of the Chemical Oxygen Demand (COD) equivalent of the supplementary carbon source to the influent nitrogen.

In some embodiments of the present invention, in step S1, the inoculated sludge is sourced from the secondary clarifier or anoxic tank of a wastewater treatment plant and is flocculent in form;

Said inoculated sludge is subjected to stagnant aeration for 12-24 hours before being seeded into the sludge acclimation and high-nitrate nitrogen wastewater denitrification treatment system, with an inoculation concentration (MLVSS) of 2000-4000 mg/L;

Said denitrifying microorganisms include Paracoccus denitrificans and Pseudomonas denitrificans, with an inoculation amount of 10³-10⁸ CFU/mL.

In some embodiments of the present invention, in step S2, the concentration of said succinic acid-producing microorganism A. succinogenes is 10⁸-10¹⁰ CFU/mL; said A. succinogenes is sourced from the China Center for Industrial Culture Collection (CICC);

The culture medium for said A. succinogenes is TSB medium;

The method further comprises washing the microorganisms 2-3 times with PBS buffer and then resuspending them in the buffer.

In some embodiments of the present invention, in step S3, the phased colonization and cultivation is divided into two stages: in the first stage, the A. succinogenes bacterial suspension is added in a pure batch mode, with an addition amount of 2×10⁸-2×10⁹ CFU/mL, added 1-3 times; in the second stage, the A. succinogenes bacterial suspension is added in a pure batch or fed-batch mode, with the addition amount gradually decreasing from 10¹² CFU/mL to 107 CFU/mL, and the number of additions corresponding to the number of cultivation cycles in the colonization period.

The conditions for said phased colonization and cultivation are a temperature of 20-35° C., a stirring speed of 80-180 rpm, and a pH of 7.0-8.5;

The total duration of said phased colonization and cultivation is 7-12 days;

The influent nitrate nitrogen concentration of the high-nitrate nitrogen wastewater in the system is 200-250 mg/L, and the C/N ratio of the supplementary carbon source is 4.2-4.8.

In some embodiments of the present invention, in step S4, the process of gradually increasing the influent nitrate nitrogen concentration of the system described in S3 is carried out in 4 cycles. The nitrate nitrogen concentrations for the 4 cycles are sequentially 200-250 mg/L, 450-500 mg/L, 700-750 mg/L, and 950-1000 mg/L, corresponding to a volumetric loading rate of 0.87-4.33 kg·m⁻³·d⁻¹. Each cycle runs for 8-12 days;

During the long-term stable operation at the maximum load, the influent nitrate nitrogen concentration is 950-1000 mg/L, the C/N ratio of the supplementary carbon source is 4.2-4.8, and the duration of operation is determined by the effluent quality and the morphology of the denitrifying granular sludge, i.e., until the effluent remains stable and meets the relevant discharge standards, and the particle size of the denitrifying granular sludge no longer increases.

In some embodiments of the present invention, in step S5, the effluent is considered stable and compliant for discharge when the total nitrogen (TN) is <35 mg/L and the ammonia nitrogen is <15 mg/L.

In some embodiments of the present invention, said granular sludge is yellowish-brown, its surface is covered with bacilli and cocci without filamentous bacteria, the bacteria are tightly adhered, the center of the granule has concave pores, and pores and honeycomb-like structures are distributed throughout the entire granule.

In some embodiments of the present invention, the diameter of said granular sludge is 980-2000 m;

The settling velocity of a single sludge granule is 176-247 m/h.

A second objective of the present invention is to provide a method for enhancing the denitrification of high-nitrate nitrogen wastewater by colonizing A. succinogenes into the activated sludge system. This utilizes its promoting effect on denitrifying microorganisms, enabling the sludge to adapt more quickly to the high-nitrate nitrogen wastewater environment, enhancing the denitrification effect, and allowing the effluent to achieve stable, compliant discharge more rapidly. Concurrently, the granular sludge formed according to the first objective of the invention further enhances the denitrification effect due to its excellent settling properties and stable microbial community structure.

The Actinobacillus succinogenes (A. succinogenes) used in the present invention is a Gram-negative, facultatively anaerobic, heterotrophic bacterium capable of efficiently producing succinic acid by metabolizing various sugars and can survive in a variety of environments.

Mechanism of Rapid Formation of Denitrifying Granular Sludge: In the present invention, after colonizing A. succinogenes into the denitrifying sludge, A. succinogenes tightly adheres to surrounding bacteria through its own high adhesiveness. It also stimulates itself and the bacteria in the sludge to secrete large amounts of extracellular polymeric substances (EPS), such as proteins and polysaccharides, which act as a “glue” between bacteria, promoting their aggregation and adhesion. At the same time, the secreted EPS provides structural support within the granular sludge framework, making the sludge granules more stable and robust, thereby enhancing their resistance to shock and durability. Furthermore, the colonization of A. succinogenes increases the hydrophobicity of the sludge and increases the Zeta potential, reducing the repulsive forces between bacteria and facilitating their agglomeration. On the other hand, the metabolic activities of A. succinogenes can produce various organic acids (such as succinic acid, acetic acid, pyruvic acid) and other metabolites. These substances can serve as carbon and energy sources for other microorganisms, promoting synergistic metabolism within the microbial community. This metabolic synergy accelerates the formation of granular sludge, making the microbial community more stable and efficient.

Mechanism of Enhancing High-Nitrate Nitrogen Wastewater Treatment: In the present invention, after colonizing A. succinogenes into the denitrifying sludge, A. succinogenes can rapidly and efficiently metabolize sugar-based carbon sources to produce small-molecule organic acids (such as succinic acid, acetic acid, pyruvic acid). These organic acids, as premium carbon sources, can be quickly utilized by denitrifying bacteria, significantly improving the efficiency of the denitrification process and enhancing the nitrogen removal effect. Additionally, by forming a stable granular sludge structure, A. succinogenes provides a stable ecological niche where denitrifying bacteria can effectively grow and metabolize in an anaerobic environment. The anaerobic microenvironment within the granular sludge is a necessary condition for denitrification, and this stable structure helps to improve denitrification efficiency and enhance the treatment of high-nitrate nitrogen wastewater.

The technical solution of the present invention possesses the following advantages over the prior art:

In the present invention, by colonizing the succinic acid-producing microorganism A. succinogenes into denitrifying sludge, the high adhesiveness of A. succinogenes and its promoting effect on denitrifying microorganisms lead to the rapid formation of denitrifying granular sludge. This simultaneously enhances the denitrification treatment of high-nitrate nitrogen wastewater, reduces the accumulation of the intermediate product nitrite, and ensures the nitrogen content of the effluent meets the Grade C discharge standard of the “Water Quality Standard for Wastewater Discharged into Urban Sewers” (GB/T 31962-2015). The method is also simple to operate and free from biological risks.

The full-process denitrifying granular sludge formed by the present invention has excellent settling properties, which can shorten the sludge settling time and increase the operational load. Furthermore, the mature denitrifying granular sludge exhibits tight adhesion between bacteria, and its entire surface is covered with pores and honeycomb-like structures. This facilitates the release of gases (such as N₂) produced during denitrification, preventing granule disintegration due to gas impact and enabling the stable maintenance of high denitrification activity.

The method of the present invention reduces the high C/N demand typically required for denitrification using glucose as a carbon source, thereby lowering the cost of adding organic carbon sources. Additionally, the method does not require pH adjustment, which reduces the addition of acid and alkali reagents, shortens the process flow, and decreases costs, making it possible to achieve energy savings and efficiency gains in the treatment of high-nitrate nitrogen wastewater.

### BRIEF DESCRIPTION OF THE DRAWINGS

To make the content of the present invention more clearly understood, the invention will be further described in detail below with reference to specific embodiments and the accompanying drawings.

FIG. 1 is a schematic flowchart of the method of the present invention.

FIG. 2 is a schematic diagram of the sludge acclimation and high-nitrate nitrogen wastewater denitrification treatment system in an embodiment.

FIG. 3 shows the change in nitrate nitrogen in the denitrification treatment system in an embodiment.

FIG. 4 shows the change in nitrite nitrogen in the denitrification treatment system in an embodiment.

FIG. 5 shows the change in total nitrogen in the denitrification treatment system in an embodiment.

FIG. 6 shows the change in effluent pH in an embodiment.

FIG. 7 shows observation images of the denitrifying sludge granulation process in an embodiment.

FIG. 8 is a Scanning Electron Microscope (SEM) image of mature denitrifying granular sludge in an embodiment.

FIG. 9 is a Transmission Electron Microscope (TEM) image of denitrifying granular sludge from reactors R1 and R0 in an embodiment.

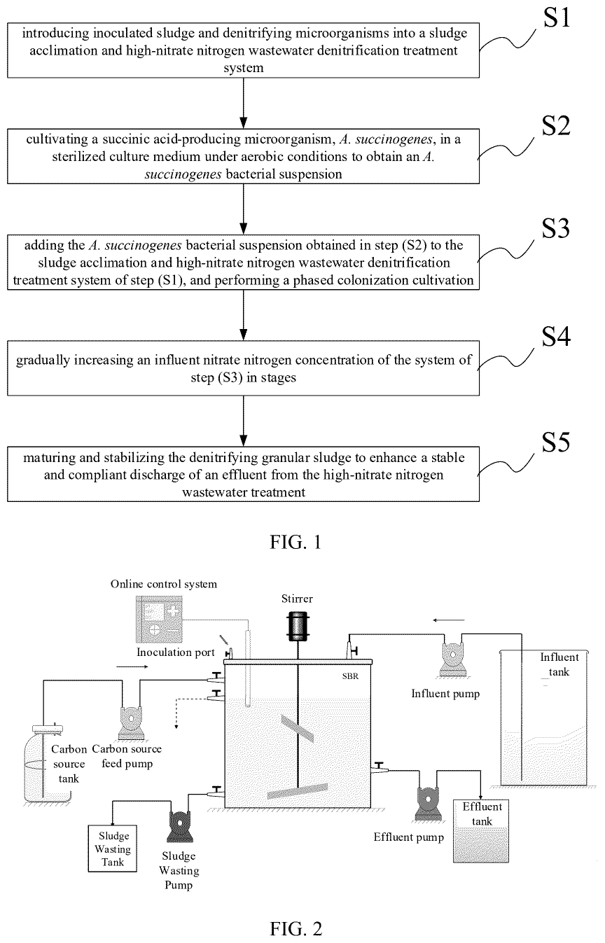

### DETAILED DESCRIPTION

The present invention will be further described below in conjunction with the accompanying drawings and specific embodiments so that those skilled in the art can better understand and implement the present invention; however, the cited embodiments are not to be construed as limiting the present invention.

When numerical ranges are given in the embodiments, it should be understood that, unless otherwise specified in the present invention, both endpoints of each numerical range, as well as any value between the two endpoints, may be selected. Unless otherwise defined, all technical and scientific terms used herein have the same meaning as commonly understood by one of ordinary skill in the art to which this invention belongs. Any methods, devices, and materials from the prior art that are similar or equivalent to those described in the embodiments of the present invention may be used to practice the present invention.

### Example 1

This embodiment provides a method for the rapid formation of denitrifying granular sludge and for enhancing the denitrification of high-nitrate nitrogen wastewater, comprising the following steps:

S1. Providing a sludge acclimation and high-nitrate nitrogen wastewater denitrification treatment system (as shown in FIG. 2) and adding inoculated sludge and the denitrifying microorganisms Paracoccus denitrificans and Pseudomonas denitrificans at an inoculation amount of 107 CFU/mL.

S2. Preparing an A. succinogenes bacterial suspension: The succinic acid-producing microorganism A. succinogenes is pre-cultured in a sterilized TSB medium under aerobic conditions to a concentration of 10¹⁰ CFU/mL, washed 3 times with 10 mM PBS, and then resuspended in 10 mL of PBS buffer to obtain the A. succinogenes bacterial suspension.

S3. Colonizing A. succinogenes in the inoculated sludge and rapidly forming denitrifying granular sludge: The A. succinogenes bacterial suspension prepared in step S2 is added to the wastewater denitrification treatment system from step S1 for colonization and cultivation under the following conditions: temperature of 20-35° C., stirring speed of 80-180 rpm, and pH of 7.0-8.5.

S4. Increasing the denitrification load in stages and achieving stable operation under high load: The influent nitrate nitrogen concentration of the wastewater denitrification treatment system is gradually increased in stages to raise the treatment load, causing the particle size of the denitrifying granular sludge to progressively increase. After reaching the maximum design denitrification load, the wastewater denitrification treatment system is maintained in long-term stable operation at the maximum load.

S5. Maturation and stabilization of the denitrifying granular sludge, enhancing the stable and compliant discharge of the effluent from the high-nitrate nitrogen wastewater treatment.

As shown in FIG. 2, the sludge acclimation and high-nitrate nitrogen wastewater denitrification treatment system in this embodiment is a conventional biological treatment system common in the art, provided that it can fulfill the biological denitrification function. It mainly includes: an influent tank, a carbon source tank, a Sequencing Batch Reactor (SBR) equipped with a stirring device, an online controller, an influent peristaltic pump, a carbon source peristaltic pump, an effluent peristaltic pump, a sludge wasting peristaltic pump, an effluent tank, a sludge wasting tank, and a bacterial suspension dosing unit. The online controller is connected to the SBR via detection probes, while the remaining devices are connected via peristaltic tubing.

The inoculated sludge in this embodiment was sourced from the secondary clarifier of a wastewater treatment plant and was flocculent in form. The raw sludge was subjected to stagnant aeration for 18 hours before being inoculated into the Sequencing Batch Reactor, with an initial sludge inoculation concentration (MLVSS) of 3000 mg/L.

The high-nitrate nitrogen wastewater in this embodiment was pre-treated steel pickling wastewater from a steel plant, with a nitrate nitrogen concentration of 200-1000 mg/L, ammonia nitrogen of 20 mg/L, total phosphorus of 10 mg/L, calcium ions of 100 mg/L, magnesium ions of 5 mg/L, fluoride ions of 2.5 mg/L, iron ions of 1 mg/L, and a pH of 7.2-7.8.

The supplementary carbon source for the wastewater denitrification system in this embodiment was glucose, and the C/N ratio was set to 4.5.

The operation of the Sequencing Batch Reactor in this embodiment consisted of four steps: influent feeding, mixing, settling, and decanting. The mixing time was 3.5 hours, the Hydraulic Retention Time (HRT) was 4 hours, the effective working volume was 4.5 L, and the system temperature was maintained at 25-30° C.

In step S3 of this embodiment, the colonization and cultivation of A. succinogenes was divided into two stages, running for a total of 40 cycles (equivalent to 6.4 days). In the first stage, a single dose of 2×10⁹ CFU/mL of the A. succinogenes bacterial suspension from step S2 was added at the same time as the inoculation of sludge and denitrifying microorganisms into the SBR. In the second stage, the A. succinogenes bacterial suspension from step S2 was added concurrently with the influent of each cycle, with the dosage gradually decreasing from 10¹³ CFU/day to 10⁸ CFU/day. No sludge was wasted during the A. succinogenes colonization phase.

During the A. succinogenes colonization phase in this embodiment, the influent wastewater had a nitrate nitrogen concentration of 200 mg/L, and the supplementary carbon source C/N ratio was 4.5.

The step of increasing the denitrification load in stages was carried out in 4 phases (as shown in Table 1). The influent nitrate nitrogen concentration was sequentially increased from 200 mg/L to 450 mg/L, 700 mg/L, and 1000 mg/L, corresponding to nitrate nitrogen volumetric loading rates of 0.87 kg·m⁻³·d⁻¹, 1.95 kg·m⁻³·d⁻¹, 3.03 kg·m⁻³·d⁻¹, and 4.33 kg·m⁻³·d⁻¹, respectively. Each phase ran for 60 cycles (10 days, with each cycle being 4 hours).

The operating conditions for each phase in this embodiment are shown in Table 1.

TABLE 1

| | | | | Volumetric |
|---|---|---|---|---|
| | Influent NO₃⁻- | Influent COD | | loading rates |
| | N (mg/L) | (mg/L) | Cycle | KgNO₃⁻-(m³d) |
| Stage | 200 | 900 | 0-40 | 0.87 |
| I | 200 | 900 | 41-100 | 0.87 |
| II | 450 | 2025 | 101-160 | 1.95 |
| III | 700 | 3150 | 161-220 | 3.03 |
| IV | 1000 | 4500 | 221-300 | 4.33 |

During the gradual increase in the particle size of the denitrifying granular sludge, the settling time was progressively shortened to eliminate poorly settling sludge, thereby promoting the formation and growth of denitrifying granules. Sludge was periodically wasted via the sludge wasting pump to maintain the sludge volume in the reactor (measured as SVI₅) at 18.2% of the reactor system's volume.

In the stable operation step under high load, the influent nitrate nitrogen concentration was 1000 mg/L, the supplementary carbon source C/N ratio was 4.5, and the operation was run for 20 cycles (equivalent to 3.3 days).

The experimental group R1 in this embodiment was the group where steps S1-S5 were fully implemented. The control group R0 was the group where A. succinogenes was not added for colonization and cultivation in step S3, while all other operational steps were identical to those of the experimental group R1.

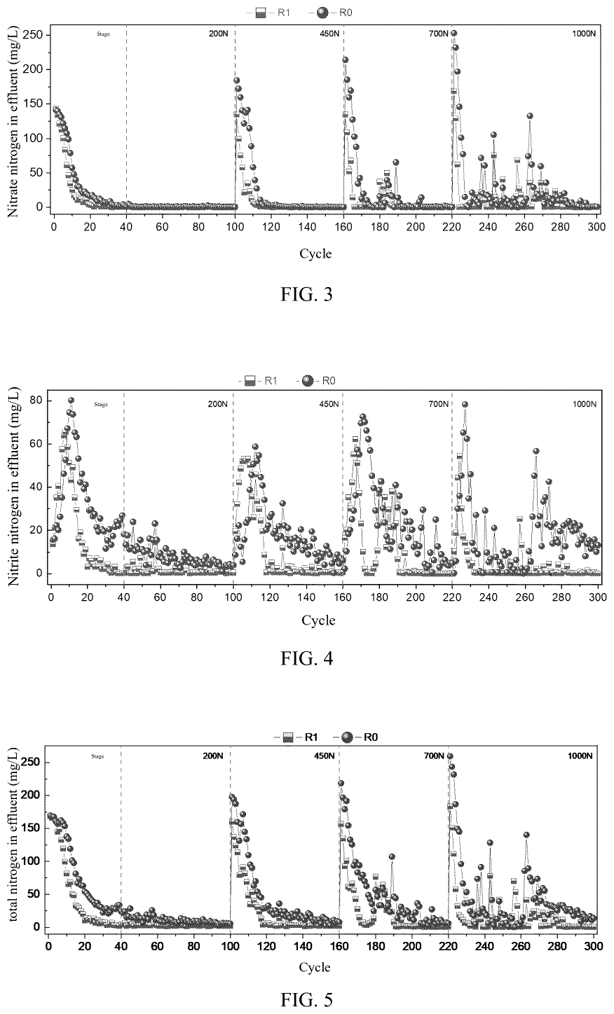

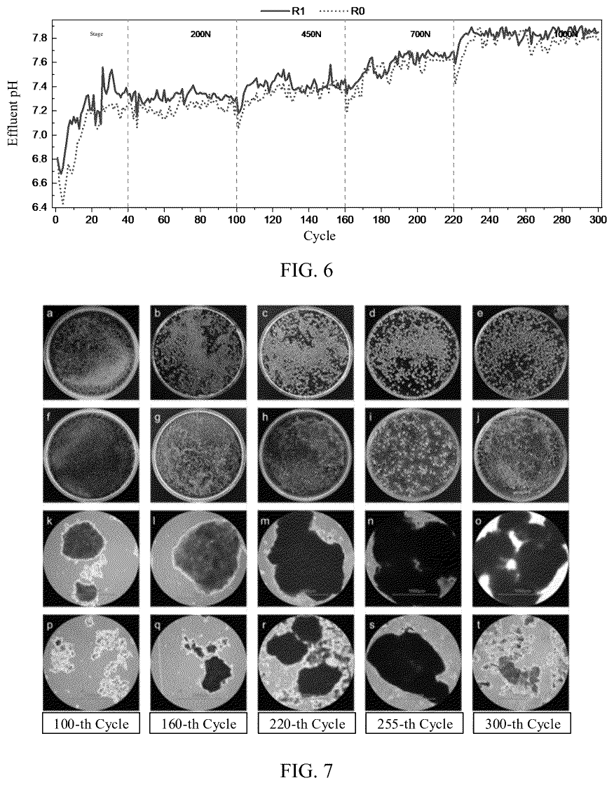

Results: As can be seen from the experimental data in FIG. 3 to FIG. 6, the experimental group R1, with the addition and colonization of A. succinogenes, exhibited stronger adaptability to the high-nitrate nitrogen environment compared to the control group R0, which did not receive A. succinogenes. In each cultivation stage, the effluent total nitrogen of R1 reached the discharge standard more quickly, which can significantly shorten the sludge acclimation period. After the effluent stabilized, the total nitrogen in the effluent of R1 was maintained below 5 mg/L, with a removal rate stably above 99%. Furthermore, the stabilized effluent contained virtually no nitrite, and the effluent ammonia nitrogen was maintained below 2 mg/L. The entire process did not require pH adjustment, and the effluent pH was maintained below 8.0. After colonization with A. succinogenes, the nitrogen content of the effluent met the Grade C discharge standard of the “Water Quality Standard for Wastewater Discharged into Urban Sewers” (GB/T 31962-2015) and the direct discharge standard of the “Discharge Standard of Water Pollutants for Iron and Steel Industry” (GB 13456-2012). Table 2 shows a comparison of effluent total nitrogen, nitrate nitrogen, nitrite nitrogen, and sludge particle size between the experimental group and the control group at the midpoint of each cultivation stage.

TABLE 2

| | Effluent TN | Effluent NO₃⁻- | Effluent NO₂⁻- | Sludge Particle |
|---|---|---|---|---|
| | (mg/L) | N (mg/L) | N (mg/L) | Size (μm) |
| R0-20 | 54.29 | 15.88 | 34.26 | 178 |
| R0-70 | 7.82 | 0.65 | 4.36 | 299 |
| R0-140 | 14.20 | 1.23 | 12.22 | 509 |
| R0-200 | 15.77 | 1.98 | 13.56 | 715 |
| R0-274 | 35.18 | 13.01 | 21.57 | 401 |
| R1-20 | 10.68 | 3.26 | 3.16 | 349 |
| R1-70 | 2.05 | 0.16 | 0.426 | 687 |
| R1-140 | 3.37 | 0.35 | 1.69 | 823 |
| R1-200 | 1.45 | 0.14 | 0.66 | 1234 |
| R1-274 | 2.34 | 1.11 | 0.61 | 1145 |

From the morphological changes of the denitrifying granular sludge shown in FIG. 7 (where a-e/k-o represent the experimental group R1, and f-j/p-t represent the control group R0), it is evident that colonization with A. succinogenes enabled faster formation of denitrifying granular sludge, which did not disintegrate after long-term stable operation at the maximum load. The mature denitrifying granular sludge was yellowish-brown with concave pores in the center, a particle diameter of 1200-2000 m, and a single granule settling velocity of 191-247 m/h. In contrast, the control group R0 formed denitrifying granular sludge at a slower rate, the particle size was smaller, and disintegration occurred after long-term operation under high load. Combined with the SEM image in FIG. 8, it can be seen that the mature denitrifying granular sludge obtained in Example 1 has a surface covered with bacilli and cocci, without filamentous bacteria. The bacteria are tightly adhered, and pores and honeycomb-like structures are distributed throughout the entire granule, providing channels for the release of gases produced during denitrification and preventing sludge disintegration caused by gas scouring.

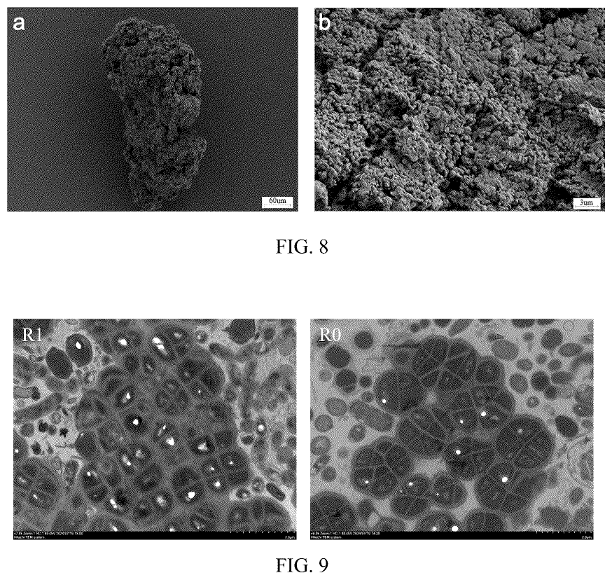

As shown in the TEM images of the denitrifying granular sludge in FIG. 9, the cell aggregates in the mature denitrifying granular sludge from Example 1 (R1) are significantly larger and contain more cells compared to the control group R0, and the cells are tightly enveloped by secreted extracellular polymeric substances.

It is apparent that the above-described embodiments are merely examples for the purpose of clear illustration and are not intended to limit the scope of the invention. For a person of ordinary skill in the art, other variations or modifications in different forms can be made based on the above description. It is not necessary or possible to exhaust all embodiments here. Any obvious variations or modifications derived therefrom still fall within the protection scope of the present invention.

1. A method for forming denitrifying granular sludge and enhancing denitrification of nitrate nitrogen wastewater, comprising the steps of:
(S1) introducing inoculated sludge and denitrifying microorganisms into a sludge acclimation and nitrate nitrogen wastewater denitrification treatment system;
(S2) cultivating a succinic acid-producing microorganism, A. succinogenes, in a sterilized culture medium under aerobic conditions to obtain an A. succinogenes bacterial suspension;
(S3) adding the A. succinogenes bacterial suspension obtained in step (S2) to the sludge acclimation and nitrate nitrogen wastewater denitrification treatment system of step (S1), and performing a phased colonization cultivation;
(S4) gradually increasing an influent nitrate nitrogen concentration of the system of step (S3) in stages, wherein a particle size of the denitrifying granular sludge progressively increases; and after reaching a maximum design denitrification load, maintaining the system of step (S3) in stable operation at the maximum load; and
(S5) maturing and stabilizing the denitrifying granular sludge to enhance a stable and compliant discharge of an effluent from the nitrate nitrogen wastewater treatment.

2. The method of claim 1, wherein in step (S1), the nitrate nitrogen wastewater has a nitrate nitrogen concentration of 200 to 1400 mg/L, an ammonia nitrogen concentration of 5 to 50 mg/L, a total phosphorus concentration of 5 to 30 mg/L, a calcium ion concentration of 0.5 to 150 mg/L, a magnesium ion concentration of 0.1 to 7.5 mg/L, a fluoride ion concentration of 0 to 7.5 mg/L, an iron ion concentration of 0.01 to 1.5 mg/L, and a pH of 7.0 to 8.0;
and wherein a supplementary carbon source for the denitrification treatment is glucose, and a C/N ratio is 3.8 to 5.5.

3. The method of claim 1, wherein in step (S1), the inoculated sludge is sourced from a secondary clarifier or an anoxic tank of a wastewater treatment plant and is flocculent in form;
wherein said inoculated sludge is subjected to stagnant aeration for 12 to 24 hours before being seeded into the sludge acclimation and nitrate nitrogen wastewater denitrification treatment system, with an inoculation concentration (MLVSS) of 2000 to 4000 mg/L; and
wherein said denitrifying microorganisms comprise Paracoccus denitrificans and Pseudomonas denitrificans, with an inoculation amount of 10³ to 10⁸ CFU/mL.

4. The method of claim 1, wherein in step (S2), a concentration of said A. succinogenes is 10⁸ to 10¹⁰ CFU/mL;
wherein said A. succinogenes is sourced from the China Center for Industrial Culture Collection;
wherein a culture medium for said A. succinogenes is TSB medium; and
wherein the method further comprises washing the A. succinogenes 2 to 3 times with a PBS buffer and resuspending in the buffer.

5. The method of claim 1, wherein in step (S3), the phased colonization cultivation is divided into two stages: a first stage wherein the A. succinogenes bacterial suspension is added in a batch mode at a dosage of 2×10⁸ to 2×10⁹ CFU/mL for 1 to 3 times; and a second stage wherein the A. succinogenes bacterial suspension is added in a batch or fed-batch mode, with a dosage gradually decreasing from 10¹² CFU/mL to 107 CFU/mL, and a number of additions corresponding to a number of cultivation cycles;
wherein conditions for said phased colonization cultivation comprise a temperature of 20 to 35° C., a stirring speed of 80 to 180 rpm, and a pH of 7.0 to 8.5;
wherein a total duration of said phased colonization cultivation is 7 to 12 days;
wherein an influent nitrate nitrogen concentration of the nitrate nitrogen wastewater in the system is 200 to 250 mg/L; and
wherein a C/N ratio of a supplementary carbon source for the nitrate nitrogen wastewater is 4.2 to 4.8.

6. The method of claim 1, wherein in step (S4), the gradual increase of the influent nitrate nitrogen concentration of the system of step (S3) is carried out over 4 cycles, wherein the nitrate nitrogen concentrations for the 4 cycles are sequentially 200 to 250 mg/L, 450 to 500 mg/L, 700 to 750 mg/L, and 950 to 1000 mg/L, corresponding to a volumetric loading rate of 0.87 to 4.33 kg·m⁻³·d⁻¹, and each cycle runs for 8 to 12 days;
and wherein during the long-term stable operation at the maximum load, the influent nitrate nitrogen concentration is 950 to 1000 mg/L, and the C/N ratio of a supplementary carbon source is 4.2 to 4.8, and a duration of the operation is determined based on effluent quality indicators and a morphology of the denitrifying granular sludge, such that the effluent meets discharge standards and the particle size of the denitrifying granular sludge no longer increases.

7. The method of claim 1, wherein in step (S5), the effluent is stable and compliant for discharge when a Total Nitrogen (TN) of the effluent is less than 35 mg/L and an ammonia nitrogen of the effluent is less than 15 mg/L.

8. A denitrifying granular sludge, prepared by a method comprising the steps of:
(S1) introducing inoculated sludge and denitrifying microorganisms into a sludge acclimation and nitrate nitrogen wastewater denitrification treatment system;
(S2) cultivating a succinic acid-producing microorganism, A. succinogenes, in a sterilized culture medium under aerobic conditions to obtain an A. succinogenes bacterial suspension;
(S3) adding the A. succinogenes bacterial suspension obtained in step (S2) to the sludge acclimation and nitrate nitrogen wastewater denitrification treatment system of step (S1), and performing a phased colonization cultivation;
(S4) gradually increasing an influent nitrate nitrogen concentration of the system of step (S3) in stages, wherein a particle size of the denitrifying granular sludge progressively increases; and after reaching a maximum design denitrification load, maintaining the system of step (S3) in long-term stable operation at the maximum load; and
(S5) maturing and stabilizing the denitrifying granular sludge to enhance a stable and compliant discharge of an effluent from the nitrate nitrogen wastewater treatment;
wherein the denitrifying granular sludge is yellowish-brown, and is cultivated and acclimated from a raw sludge sourced from a secondary clarifier or an anoxic tank of a wastewater treatment plant through colonization with the succinic acid-producing microorganism A. succinogenes;
wherein a surface of the denitrifying granular sludge is covered with bacilli and cocci without filamentous bacteria, and bacteria thereon are tightly adhered to each other; and
wherein the denitrifying granular sludge has concave pores in a central part, and pores and honeycomb-like structures are distributed throughout the entire denitrifying granular sludge.

9. The denitrifying granular sludge of claim 8, wherein a diameter of said denitrifying granular sludge is from 980 to 2000 m;
and wherein a settling velocity of a single granule of said denitrifying granular sludge is from 176 to 247 m/h.
